(19) United States
(12) Patent Application Publication
(10) Pub. No.: US 2008/0177103 A1
(43) Pub. Date: Jul. 24, 2008

(54) Biologically Degradable Gas Hydrate Inhibitors

(76) Inventors: Dirk Leinweber, Schwalbach (DE); Michael Feustel, Koengernheim (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/885,611

(22) PCT Filed: Feb. 16, 2006
(86) PCT No.: PCT/EP2006/001394

§ 371 Date: Aug. 31, 2007

(30) Foreign Application Priority Data
Mar. 1, 2005 (DE) 10 2005 009 134.2

(51) Int. Cl. C07C 229/02

(52) U.S. Cl. 560/155

(57) ABSTRACT

The invention relates to tri- and tetracarboxylic acid ester of the formula (1) wherein R¹ and R² independently of one another are C₁- to C₂₂-alkyl, C₂- to C₂₂-alkenyl, C₆- to C₃₀-aryl or C₇- to C₃₀-alkylaryl, and A is a C₂- to C₄-alkylene group, B is an arbitrarily substituted organic group having 3-100 carbon atoms, n is a number from 1 to 30, m is 3 or 4, whereby the structural units —NR¹-R² are partly or completely quaternized and the use thereof as a gas hydrate inhibitor.

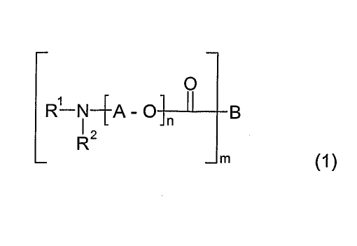

The present invention relates to tri- and tetracarboxylic acid esters of aminopolyethers, their preparation and use and a method for inhibiting nucleation, growth, and/or agglomeration of gas hydrates by adding an effective amount of an inhibitor which contains substituted polyesters to a multiphase mixture tending to form hydrates and consisting of water, gas and optionally condensate or to a drilling fluid tending to form gas hydrates.

Gas hydrates are crystalline inclusion compounds of gas molecules in water which form under certain temperature and pressure conditions (low temperature and high pressure). The water molecules form cage structures around the corresponding gas molecules. The framework formed from the water molecules is thermodynamically unstable and is stabilized only by incorporation of guest molecules. These ice-like compounds can exist even above the freezing point of water (up to more than 25° C.), depending on pressure and gas composition.

In the mineral oil and natural gas industry, in particular the gas hydrates which form from water and the natural gas constituents methane, ethane, propane, isobutane, n-butane, nitrogen, carbon dioxide and hydrogen sulfide are of considerable importance. Particularly in natural gas extraction today, the existence of these gas hydrates presents a major problem more particularly when wet gas or multiphase mixtures of water, gas and alkane mixtures are exposed to low temperatures under high pressure. Here, owing to their insolubility and crystalline structure, the formation of the gas hydrates leads to blockage of various transport facilities, such as pipelines, valves or production facilities, in which wet gas or multiphase mixtures are transported over relatively long distances at relatively low temperatures, as occurs especially in colder regions of the earth or at the bottom of the sea.

In addition, the gas hydrate formation can also lead to problems in the drilling process for developing new gas or mineral oil deposits under corresponding pressure and temperature conditions, by virtue of the fact that gas hydrates form in the drilling fluids.

In order to avoid such problems, the gas hydrate formation in gas pipelines, during transport of multiphase mixtures or in drilling fluids can be suppressed by using relatively large amounts (more than 10% by weight, based on the weight of the water phase) of lower alcohols, such as methanol, glycol or diethylene glycol. As a result of adding these additives, the thermodynamic limit of the gas hydrate formation is shifted to lower temperatures and higher pressures (thermodynamic inhibition). However, the addition of these thermodynamic inhibitors gives rise to greater safety problems (flashpoint and toxicity of the alcohols), logistical problems (large storage tanks, recycling of these solvents) and accordingly high costs, especially in offshore production.

To date, attempts have therefore been made to replace thermodynamic inhibitors by adding, in temperature and pressure ranges in which gas hydrates can form, additives in amounts of <2% which either retard the gas hydrate formation (kinetic inhibitors) or keep the gas hydrate agglomerates small and hence pumpable so that they can be transported through the pipeline (so-called agglomerate inhibitors or antiagglomerants). The inhibitors used either hinder the nucleation and/or the growth of the gas hydrate particles or modify the hydrate growth in such a way that smaller hydrate particles result.

In addition to the known thermodynamic inhibitors, the patent literature describes, as gas hydrate inhibitors, a multiplicity of monomeric as well as polymeric classes of substances which are kinetic inhibitors or antiagglomerants. Of particular importance here are polymers and their carbon backbone which contain both cyclic (pyrrolidone or caprolactam radicals) and acyclic amide structures in the side groups.

Thus, WO-94/12761 discloses a method for kinetic-inhibition of gas hydrate formation by the use of polyvinyllactams having a molecular weight of M_{w}>40 000 D, and WO-93/25798 discloses such a method using polymers and/or copolymers of vinylpyrrolidone having a molecular weight of M_{w}>5000 to 40 000 D.

EP-A-0 896 123 discloses gas hydrate inhibitors which may contain copolymers of alkoxylated methacrylic acid without an alkyl endcap and cyclic N-vinyl compounds.

EP-A-1 048 892 describes the use of additives for improving the flow of water-containing petroleum, it being possible for this to contain polyvinyl alcohol or partly hydrolyzed polyvinyl acetate as a nucleating agent for gas hydrates in combination with suitable dispersants. The document makes no further statement about the polyvinyl alcohol or the partly hydrolyzed polyvinyl acetate, apart from the fact that the molecular weight thereof should be below 50 000 g/mol.

U.S. Pat. No. 5,244,878 describes a method for retarding the formation or reducing the tendency of formation of gas hydrates. For this purpose, polyols which are esterified with fatty acids or alkenylsuccinic anhydrides are used. The compounds prepared have no amino acid functions which might interact with clathrates (cage molecules).

The additives described have only limited efficiency as kinetic gas hydrate inhibitors and/or antiagglomerants, must be used with coadditives or are obtainable in insufficient amount or only at high prices.

In order to be able to use gas hydrate inhibitors even with greater supercooling than currently possible, i.e. further within the hydrate region, there is a need for a further increase in activity in comparison with the hydrate inhibitors of the prior art. In addition, products improved with respect to their biological degradability and toxicity are desired.

It was therefore an object of the present invention to find improved additives which will both slow down the formation of gas hydrates (kinetic inhibitors) and keep gas hydrate agglomerates small and pumpable (antiagglomerants) in order thus to ensure a broad range of use with high potential activity. Furthermore, they should be capable of replacing the currently used thermodynamic inhibitors (methanol and glycols) which give rise to considerable safety problems and logistical problems.

As has now surprisingly been found, both water-soluble and oil-soluble tri- and tetracarboxylic acid esters of amino polyethers are suitable as gas hydrate inhibitors. Depending on structure, these esters can both retard the nucleation and the growth of gas hydrates (kinetic gas hydrate inhibitors) and suppress the agglomeration of gas hydrates (antiagglomerants).

The invention therefore relates to tri- and tetracarboxylic acid. esters. of the formula (I)

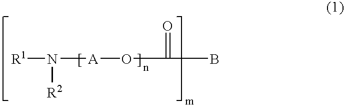

in which
R¹, R² independently of one another are C₁- to C₂₂-alkyl, C₂- to C₂₂-alkenyl, C₆- to C₃₀-aryl or C₇- to C₃₀-alkylaryl, and
A is a C₂- to C₄-alkylene group,
B is an arbitrarily substituted organic group having 3-100 carbon atoms,
n is a number from 1 to 30,
m is 3 or 4,
   it being possible for the structural units —NR¹—R² to be partly or completely quaternized.

The invention furthermore relates to a method for inhibiting the formation of gas hydrates by adding esters as defined above in amounts of from 0.01 to 2% by weight to an aqueous phase which is in contact with a gaseous, liquid or solid organic phase and in which gas hydrate formation is to be prevented.

The invention furthermore relates to the use of the tri- and tetracarboxylic acid esters according to the invention in amounts of from 0.01 to 2% by weight for preventing the formation of gas hydrates in aqueous. phases which are in contact with a gaseous, liquid or solid organic phase.

A is preferably an alkylene radical having 2 or 3 carbon atoms.

R¹ and R², independently of one another, are preferably C₃- to C₈-alkyl radicals.

n is preferably an integer from 2 to 10, in particular from 3 to 6.

B is preferably an organic radical having 2 to 10 carbon atoms, in particular having 2 to 6 carbon atoms. B preferably comprises hydroxyl groups. B particularly preferably comprises aliphatic radicals which may be saturated or unsaturated and contain 3 to 6 carbon atoms, or aromatic radicals having 6 to 10 carbon atoms.

The tri- and tetracarboxylic acid esters according to the invention can be prepared by processes known from the literature, by esterification of tri- or tetracarboxylic acids of the formula B(COOH)ₘ with alkoxylated dialkylamines of the formula R¹R²N-(A-O)ₙ—H.

According to the invention, partial or complete quaternization of the structural units —NR¹R² of the prepared tri- and tetracarboxylic acid esters can also be effected using customary alkylating agents (alkyl or aryl halides or alkyl sulfates), such as, for example, methyl chloride, benzyl chloride, sodium monochloroacetate or dimethyl sulfate.

In a preferred embodiment, the compounds of the formula (I) are based on the following tri- and tetracarboxylic acids B(COOH)ₘ or anhydrides thereof: citric acid, pyrocitric acid, trimellitic acid, pyromellitic acid, 1,2,3-benzenetricarboxylic acid, 1,3,5-benzenetricarboxylic acid, butane-1,2,3,4-tetracarboxylic acid, trimellitic anhydride, pyromellitic anhydride.

Dialkylamines having C₁- to C₂₂-alkyl radicals or C₂- to C₂₂-alkenyl radicals, preferably C₃- to C₈-dialkylamines, form the basis of the alkoxylated dialkylamines used. Suitable dialkylamines are, for example, di-n-butylamine, diisobutylamine, dipentylamine, dihexylamine, dioctylamine, dicyclopentylamine, dicyclohexylamine, diphenylamine, dibenzylamine.

The alkylamines are generally reacted with ethylene oxide, propylene oxide, butylene oxide or mixtures of differing such alkylene oxides, ethylene oxide or mixtures of ethylene oxide and propylene oxide being preferred. The alkylamines are treated with from 1 to 30 mol of alkylene oxide, preferably from 2 to 10 mol, particularly preferably from 3 to 6 mol, based on the alkylamines.

The alkoxylation is effected in the absence of a solvent but may also be carried out in solution. Suitable solvents for the alkoxylation are inert ethers, such as dioxane, tetrahydrofuran, glyme, diglyme and MPEGs.

In general, the alkoxylation is carried out in the first reaction step without catalysis up to >95% by weight of tertiary nitrogen. Higher alkoxylation is effected after addition of basic compounds as catalysts. Basic compounds which may be used are alkaline earth metal/alkali metal hydroxides or alcoholates (sodium methylate, sodium ethylate, potassium tert-butylate), but alkali metal hydroxides are preferred, particularly sodium hydroxide or potassium hydroxide.

The preparation of tri- and tetracarboxylic acid esters is known in the prior art and is effected by uncatalyzed or acid-catalyzed condensation of the respective tri- or tetracarboxylic acid with the corresponding alkoxylated dialkylamine. The reaction temperature is in general from 100 to 250° C., preferably from 120 to 150° C. The reaction can be carried out at atmospheric pressure or reduced pressure. Catalyzing acids which may be mentioned are, for example, HCl, H₂SO₄, sulfonic acids, H₃PO₄ or acidic ion exchangers, which are used in amounts of from 0.1 to 5% by weight, based on the weight of the reaction mixture. The condensation takes in general from 3 to 10 hours.

The number-average molecular weight of the tri- and tetracarboxylic acid esters according to the invention is preferably from 500 to 50 000 g/mol, particularly preferably from 1000 to 10 000 g/mol.

The tri- and tetracarboxylic acid esters can be used alone or in combination with other known gas hydrate inhibitors. In general, the gas hydrate inhibitor according to the invention is added to the system tending to form hydrates in an amount such that sufficient inhibition is obtained under the given pressure and temperature conditions. The gas hydrate inhibitors according to the invention are generally used in amounts of from 0.01 to 2% by weight (based on the weight of the aqueous phase), corresponding to 100-20000 ppm, preferably from 0.02 to 1% by weight. If the gas hydrate inhibitors according to the invention are used as a mixture with other gas hydrate inhibitors, the concentration of the mixture is from 0.01 to 2 or from 0.02 to 1% by weight in the aqueous phase.

For use as gas hydrate inhibitors, the tri- and tetracarboxylic acid esters are preferably dissolved in water-miscible alcoholic solvents, such as, for example, methanol, ethanol, propanol, butanol, ethylene glycol and oxyethylated monoalcohols, such as butyl glycol, isobutyl glycol, butyl diglycol.

### EXAMPLES

### Preparation of the Tri- and Tetracarboxylic Acid Esters

### Example 1

### Reaction of Citric Acid with Dibutylamine Tetraglycol Ether

In a 250 ml four-necked flask with stirrer, thermometer, nitrogen flushing and distillation bridge, 29 g of citric acid, 156 g of dibutylamine tetraglycol ether and 1.9 g of p-toluenesulfonic acid were mixed and heated to 140-150° C. Water was then distilled off until the acid number was not more than 5 mg KOH/g. The resulting citric acid ester had a saponification number of 155.3 mg KOH/g.

### Example 2

### Reaction of Trimellitic Anhydride with Dibutylamine Tetraglycol Ether

In a 250 ml four-necked flask with stirrer, thermometer, nitrogen flushing and distillation bridge, 32 g of trimellitic anhydride, 165 g of dibutylamine tetraglycol ether and 1.9 g of p-toluenesulfonic acid were mixed and heated to 140° C., a homogeneous reaction mixture forming. Heating to 180-200° C. was then effected in the course of 2 h and water was distilled off until the acid number was not more than 5 mg KOH/g. The resulting trimellitic acid ester had a saponification number of 148.4 mg KOH/g.

### Example 3

### Reaction of Pyromellitic Anhydride with Dibutylamine Tetraglycol Ether

In a 500 ml four-necked flask with stirrer, thermometer, nitrogen flushing and distillation bridge, 36 g of pyromellitic anhydride, 220 g of dibutylamine tetraglycol ether and 1.9 g of p-toluenesulfonic acid were mixed and heated to 140° C., a homogeneous reaction mixture forming. Heating to 180-200° C. was then effected in the course of 2 h and water was distilled off until the acid number was not more than 5 mg KOH/g. The resulting pyromellitic acid ester had a saponification number of 159.8 mg KOH/g.

### Example 4

The citric acid ester from Example 1 was quaternized with dimethyl sulfate.

### Example 5

The trimellitic acid ester from Example 2 was quaternized with dimethyl sulfate.

### Example 6

The pyromellitic acid ester from Example 3 was quaternized with dimethyl sulfate.

### Efficiency of the Polymers as Gas Hydrate Inhibitors

For investigating the inhibiting effect of the polyesters, a stirred steel autoclave with temperature control, pressure and torque sensor and with an internal volume of 450 ml was used. For investigations of the kinetic inhibition, the autoclave was filled with distilled water and gas with the volume ratio 20:80; for investigations of the agglomerate inhibition, condensate was additionally introduced. Finally, natural gas under 90 bar was forced in. Starting from an initial temperature of 17.5° C., cooling was effected to 2° C. in the course of 2 h, followed by stirring for 18 h at 2° C. and heating back to 17.5° C. in the course of 2 h. A pressure decrease according to the thermal compression of the gas was first observed. If the formation of gas hydrate nuclei occurs during the supercooling time, the measured pressure decreases, an increase in the measured torque and a slight increase in the temperature being observable. Further growth and increasing agglomeration of the hydrate nuclei rapidly lead to a further increase in the torque in the absence of inhibitor. When warming the mixture, the gas hydrates disintegrate so that the initial state of the experimental series is reached.

The time from reaching the minimum temperature of 2° C. to the first gas absorption (T_{ind}) or the time to the increase in the torque (T_{agg}) is used as a measure of the inhibiting effect of the polymer. Long induction times or agglomeration times indicate an effect as a kinetic inhibitor. On the other hand, the torque measured in the autoclave serves as a parameter for the agglomeration of the hydrate crystals. In the case of a good antiagglomerant, the torque which builds up after formation of gas hydrates is substantially reduced compared with the blank value. Ideally, snow-like, fine hydrate crystals form in the condensate phase, which hydrate crystals do no agglomerate and hence do not lead to blockage of the installations serving for gas transport and for gas extraction.

### Test Results

Composition of the natural gas used:

Methane 87.6%, ethane 1.26%, propane 0.08%, butane 0.02%, carbon dioxide 0.35%, nitrogen 10.61%.

Supercooling below the equilibrium temperature of hydrate formation at 90 bar: 8.5° C.

A commercially available gas hydrate inhibitor based on polyvinylpyrrolidone was used as a comparative substance. The metering rate was 5000 ppm, based on the water phase, in all experiments.

TABLE 1

| Ester from Example | T_{ind} (h) | T_{agg} (h) |
|---|---|---|
| Blank value | 0 | 0 |
| 1 | 9.4 | 9.4 |
| 2 | 13.2 | 13.3 |
| 3 | 15.1 | 15.1 |
| Comparison | 3.0 | 3.1 |

As is evident from the above test results, the tri- or tetraesters according to the invention act as kinetic hydrate inhibitors and exhibit a substantial improvement compared with the prior art. The compounds are biologically degradable, as shown below.

TABLE 2
(according to OECD 306)

| Ester from Example | Biological degradation in % |
|---|---|
| Comparison | 6 |
| 1 | 62 |
| 2 | 40 |
| 3 | 37 |

In order to test the effect as agglomerate inhibitors, water and mineral spirit (20% of the volume in the ratio 1:2) were initially introduced into the test autoclave used above and 5000 ppm of the respective additive were added, based on the water phase.

At an autoclave pressure of 90 bar and a stirring speed of 5000 rpm, the temperature was decreased from initially 17.5° C. in the course of 2 hours to 2° C., stirring was then effected for 16 hours at 2° C. and the temperature was increased again. During this procedure, the agglomeration time until the occurrence of gas hydrate agglomerates and the resultant torque at the stirrer, which is a measure of the agglomeration of the gas hydrates, were measured. A commercially available antiagglomerant (quaternary ammonium salt) was used as a comparative substance.

TABLE 3

| Polyester from | | |
|---|---|---|
| Example | T_{agg} (h) | Mₘₐₓ (Ncm) |
| Blank value | 0.1 | 15.9 |
| 4 | 4.1 | 2.1 |
| 5 | 2.5 | 1.2 |
| 6 | 2.9 | 1.1 |
| Comparison | 2.2 | 3.7 |

As is evident from these examples, the measured torques are greatly reduced in comparison with the blank value, in spite of gas hydrate formation. This indicates a substantial agglomerate-inhibiting effect of the products according to the invention. All examples show a substantially better performance than the commercially available antiagglomerant (comparison=prior art). The compounds are biologically degradable, as shown below.

TABLE 4
(according to OECD 306)

| Quaternized ester from Example | Biological degradation in % |
|---|---|
| Comparison | 6 |
| 4 | 35 |
| 5 | 27 |
| 6 | 25 |

1. A tri- or tetracarboxylic acid ester of formula (1) 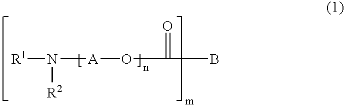
in which
R¹, R² independently of one another are C₁- to C₂₂-alkyl, C₂- to C₂₂-alkenyl, C₆- to C₃₀-aryl or C₇- to C₃₀-alkylaryl, and
A is a C₂- to C₄-alkylene,
B is an optionally substituted organic group having 3-100 carbon atoms,
n is a number from 1 to 30,
m is 3 or 4,
wherein it being possible for the structural units —NR¹—R² are partly or completely quaternized.

2. The compound as claimed in claim 1, wherein the carboxylic acid from which the ester of the formula 1 is prepared is selected from the group consisting of citric acid, pyrocitric acid, trimellitic acid, pyromellitic acid, 1,2,3-benzenetricarboxylic acid, 1,3,5-benzenetricarboxylic acid, butane-1,2,3,4-tetracarboxylic acid, trimellitic anhydride, and pyromellitic anhydride.

3. The compound as claimed in claim 1, wherein A is ethylene or propylene.

4. The compound of claim 1, wherein the structural unit —NR¹R² is partly or completely quaternized with methyl chloride, benzyl chloride, sodium monochloroacetate or dimethyl sulfate.

5. The compound of claim 1, wherein n is a number from 1 to 6.

6. A method for inhibiting gas hydrate formation in a multiphase hydrocarbon mixture comprising a gas, water, and optionally a condendate, said method comprising adding to the multiphase hydrocarbon mixture the tri- or tetracarboxylic acid ester or a combination thereof of claim 1 from 0.01 to 2% by weight, based on the mixture.
